SEQUENCE LISTING (4 sequences)

SEQ ID NO: 1 - protein, 21 aa, Artificial Sequence
  Fragment of Endothelin-1
CSCSSLMDKECVYFCHLDIIW

SEQ ID NO: 2 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 3 - protein, 16 aa, Artificial Sequence
  Fragment of GRO.alpha.
CAQTEVIATLKNGRKA

SEQ ID NO: 4 - protein, 30 aa, Artificial Sequence
  Fragment of VCAM-1
CVNLIGKNRKEVELIVQEKPFTVEISPGPR

(19) United States
(12) Patent Application Publication
(10) Pub. No.: US 2018/0299399 A1
(43) Pub. Date: Oct. 18, 2018

(54) ELECTROCHEMICAL ASSAY FOR A PROTEIN ANALYTE

(71) Applicants: UNIVERSITY HEALTH NETWORK, Toronto (CA); THE GOVERNING COUNCIL OF THE UNIVERSITY OF TORONTO, Toronto (CA)

(72) Inventors: Shafique KESHAVJEE, Toronto (CA); Mingyao LIU, North York (CA); Andrew SAGE, Toronto (CA); Edward SARGENT, Toronto (CA); Shana KELLEY, Toronto (CA)

(21) Appl. No.: 15/768,082

(22) PCT Filed: Oct. 14, 2016
(86) PCT No.: PCT/CA2016/000259

§ 371 Date: Apr. 13, 2018

Related U.S. Application Data
(60) Provisional application No. 62/241,439, filed on Oct. 14, 2015.

(51) Int. Cl. G01N 27/327; G01N 33/543; G01N 33/563; G01N 33/566

(52) U.S. Cl. CPC G01N 27/3276; G01N 27/3278; G01N 33/5438; B82Y 30/00; G01N 33/566; G01N 2800/60; G01N 2800/56; G01N 33/563

(57) ABSTRACT

There is described herein a method for the electrochemical quantification of a protein analyte in sample, comprising: providing one or more electrode(s), each comprising at least one peptide attached to its surface, the peptide being the protein or a fragment thereof; contacting the sample and electrode with an antibody in the presence of a redox reporter, wherein the antibody is capable of binding to each of the protein analyte and the peptide on the electrode; measuring an electrochemical signal generated by the redox reporter when a potential is applied; quantifying the protein analyte by comparing the electrochemical signal generated with a control, wherein the electrochemical signal is indirectly proportional to the amount of protein analyte in the sample.

### FIELD OF THE INVENTION

The invention relates to electrochemical assays, and more particularly electrochemical assays for detecting or quantifying a protein in a sample.

### BACKGROUND OF THE INVENTION

There are numerous clinical and laboratory settings that require protein detection or quantitation. Depending on the accuracy and speed required and the amount and purity of the protein available, different methods are appropriate for determining protein presence and concentration.

Lung transplantation (LTx) is a life-saving procedure for patients suffering from end-stage lung disease. At present, donor lungs are assessed for transplant suitability based on several physiological parameters including donor/organ medical history and pulmonary compliance measures. These physiological metrics do not reliably predict recipient outcomes after transplant. Thus, the inclusion of lung-specific biomarker tests, prior to transplantation, that could accurately predict LTx outcomes would be of great benefit to patients and transplant teams.

Ex-vivo lung perfusion (EVLP) is a novel technique that has been developed to improve the LTx procedure by affording more time for transplant teams to assess and treat a donor lung under normothermic conditions¹. As such, EVLP can provide a means by which donor lungs can be treated therapeutically without the detrimental effects of the host immune system¹. In addition, EVLP can allow for the discovery, validation, and monitoring of predictive biomolecules in EVLP perfusate. In studies using EVLP, circulating levels of the endothelin-1 (ET-1) peptide have been shown to be predictive of donor lung function².

ET-1 is an important chemokine that plays a key role in vasoconstriction and fibroblast proliferation³⁻⁵. The effects of increased ET-1 expression have been implicated as a significant risk factor for both acute and chronic lung injury. Primary graft dysfunction (PGD) is a severe form of acute rejection and can occur in approximately 30% of LTx cases. Recent work has demonstrated strong correlation between ET-1 levels and the development of PGD through the disruption of the alveolar-capillary barrier². The profibrotic properties of ET-1 are also a significant contributor to the narrowing of the bronchioles which represents a major characteristic of chronic lung allograft dysfunction (CLAD)^{6,7} Bronchiolitis obliterans syndrome (BOS) is the predominant form of CLAD and is the principal cause of late graft loss⁸. ET-1 concentrations have been shown to correlate with the development of BOS⁹. Therefore, ET-1 is an extremely powerful biomarker that can be used to predict short- and long-term survival in transplant patients and is a valuable target for molecular diagnostics. There are a number of similar biomarkers that have been shown to be important in measuring risk acute lung injury (see for example, U.S. Patent Publication No. 2015/0377904).

Current detection strategies for ET-1 are based primarily on the enzyme-linked immunosorbent assay (ELISA) protocol^{2, 9, 10}. The typical workflow of an ET-1 ELISA can take upwards of 4 hours and requires significant user input. Yet, to be clinically relevant within the decision-making processes of LTx¹¹, assays must provide sample-to-answer times that are much faster than a typical ELISA. As such, integration of ET-1 testing into the transplant setting remains problematic.

### SUMMARY OF THE INVENTION

In an aspect, there is provided a method for the electrochemical quantification of a protein analyte in sample, comprising: providing one or more electrode(s), each comprising at least one peptide attached to its surface, the peptide being the protein or a fragment thereof; contacting the sample and electrode with an antibody in the presence of a redox reporter, wherein the antibody is capable of binding to each of the protein analyte and the peptide on the electrode; measuring an electrochemical signal generated by the redox reporter when a potential is applied; quantifying the protein analyte by comparing the electrochemical signal generated with a control, wherein the electrochemical signal is indirectly proportional to the amount of protein analyte in the sample.

In an aspect, there is provided a kit for the electrochemical detection of a protein analyte in sample, the kit comprising: an electrode comprising a peptide attached to its surface, the peptide being the protein or a fragment thereof; an antibody capable of binding to the protein analyte and the peptide on the electrode; a redox reporter; and instructions for use.

### BRIEF DESCRIPTION OF FIGURES

These and other features of the preferred embodiments of the invention will become more apparent in the following detailed description in which reference is made to the appended drawings wherein:

FIG. 1 shows ET-1 analysis assay (EAA). (a) Schematic representation of the EAA microchip showing the cross-sectional components of the microchip (dashed red box). A thin layer of gold is deposited on a glass substrate and passivated with SU-8 using photolithography to create gold apertures for sensor electrodeposition. (b) An SEM image of an electrodeposited sensor. The scale bar is indicated in the lower right corner of the image. (c) Sensors (yellow) are functionalized via the N-terminal cysteine residue of the ET-1 peptide (blue). With low levels of ET-1 peptide present in EVLP perfusate (upper track), the addition of ET-1 antibodies (pink) bind and sterically hinder the electrode surface during the oxidation of ferrocyanide (red). Conversely, high levels of endogenous ET-1 in EVLP perfusate (lower track) bind ET-1 antibodies that would otherwise bind the ET-1 peptide on the electrode surface, thus reducing the steric hindrance of electron transfer at the electrode surface.

FIG. 2 shows validation of ET-1 detection scheme. (a) Differential pulse voltammograms for various degrees of sensor blocking arising from: no antibody (solid line), ET-1 antibodies (dashed line), and both ET-1 peptide and antibodies (dotted line) in solution. (b) Representative quantifications of DPV currents with and without antibody or ET-1 peptide present in solution. Each point represents n=20 different sensors and error-bars indicate s.e.m. (c) Currents obtained (reported as % available surface) for the oxidation of ferrocyanide at the electrode surface for various concentrations of ET-1 antibody bound to ET-1 peptide SAM (1 ng/mL). Each point represents n=20 different sensors and error-bars indicate s.e.m. (d) OD 450 nm measurements for various concentrations of endogenous ET-1 using the competitive ELISA-based technique. r² represents the goodness of fit using non-linear regression. The concentration of ET-1 antibody was 1 μg/mL.

FIG. 3 shows ET-1 detection in EVLP. (a) Currents obtained (reported as available surface) for the oxidation of ferrocyanide at the electrode surface for the detection of ET-1 in STEEN solution. The equation and goodness of fit for the standard curve (circles, solid line) are shown in the upper left quadrant of the graph. Each point represents n=5 different sensors and error-bars indicate s.e.m. The dashed lines represent the observed % available surface for two spiked ET-1 concentrations, x₁ and x₂, extrapolated to theoretical anti-ET-1 concentrations. (b) Calculated ET-1 concentrations using the EAA in perfusate samples collected from a donor lung during EVLP. Each point represents n>3 sensors and error-bars indicate s.e.m.

FIG. 4 shows indirect detection of GROα using peptide SAM and targeted antibodies. Gold biosensors are functionalized with antibody specific peptides from a fragment of GROα to form a self-assembled monolayer (SAM) (Left). A known concentration of antibody is then incubated with target sample (Middle) and then hybridized with the peptide-functionalized sensors (Right). Antibody and protein complexes are unable to bind the sensor and are subsequently washed away leaving only unbound antibody on the surface of the electrode. The concentration of target protein can then be calculated based on the amount of antibody detected on the surface of the sensor.

FIG. 5 shows validation of GROα detection scheme. Signals obtained without GROα or GROα antibody (−/− (left)) compared to the signal from the anti-GROα antibody only (+/−(middle)) and when the concentration of GROα in solution is equal to the GROα antibody concentration (+/+(right)). Data represented are mean+/−s.e.m. (n=3).

FIG. 6 shows indirect detection of VCAM-1 using modified-peptide SAM and targeted antibodies. A) Antigenic 20 amino acid peptide used for the generation of a VCAM-1 antibody. B) Thiol modification of the VCAM-1 peptide. C) Gold biosensors are functionalized with thiol-modified VCAM-1 peptides to form a self-assembled monolayer (SAM) (Left). A known concentration of antibody is then incubated with target sample (Middle) and then hybridized with the peptide-functionalized sensors (Right). Antibody and protein complexes are unable to bind the sensor and are subsequently washed away leaving only unbound antibody on the surface of the electrode. The concentration of target protein can then be calculated based on the amount of antibody detected on the surface of the sensor.

FIG. 7 shows validation of VCAM-1 detection scheme. Signals obtained without VCAM-1 protein or VCAM-1 antibody (0% Blocking (left)) compared to the signal when the concentration of VCAM-1 in solution is half to the VCAM-1 antibody concentration (50% Blocking (middle)) and from the anti-VCAM-1 antibody only (100% Blocking (right)). Data represented are mean+/−s.e.m. (n=3).

FIG. 8 shows ET-1 detection for EVLP.

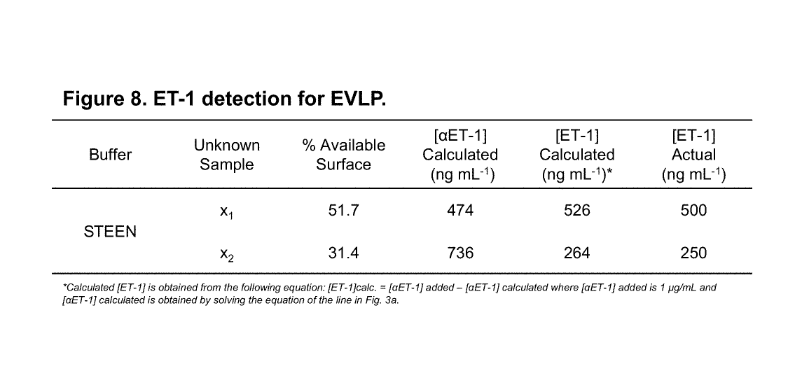

FIG. 9 shows ET-1 biosensor characteristics. Currents obtained (reported as available surface for various concentrations of ET-1 antibody bound to the ET-1 peptide SAM (1 ng/mL) for sensors that were electrodeposited for 30 (triangles), 60 (inverted triangles), or 120 (circles) seconds.

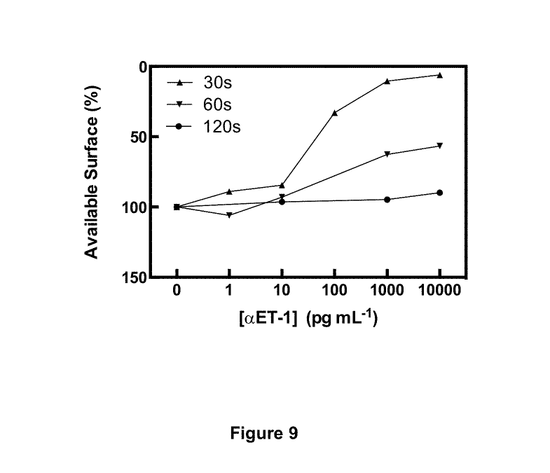

FIG. 10 shows ET-1 detection in PBS. Currents obtained (reported as % available surface) for the oxidation of ferrocyanide at the electrode surface for the detection of ET-1 in PBS. The equation and goodness of fit for the standard curve (circles, solid line) are shown in the upper left quadrant of the graph. Each point represents n=5 different sensors and error-bars indicate s.e.m. The dashed lines represent the observed % available surface for two unknown ET-1 concentrations, x₁ and x₂, extrapolated to theoretical anti-ET-1 concentrations.

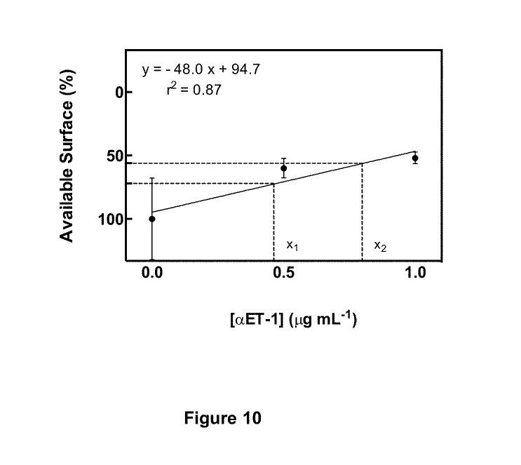

FIG. 11 shows Estimated ET-1 concentrations in PBS.

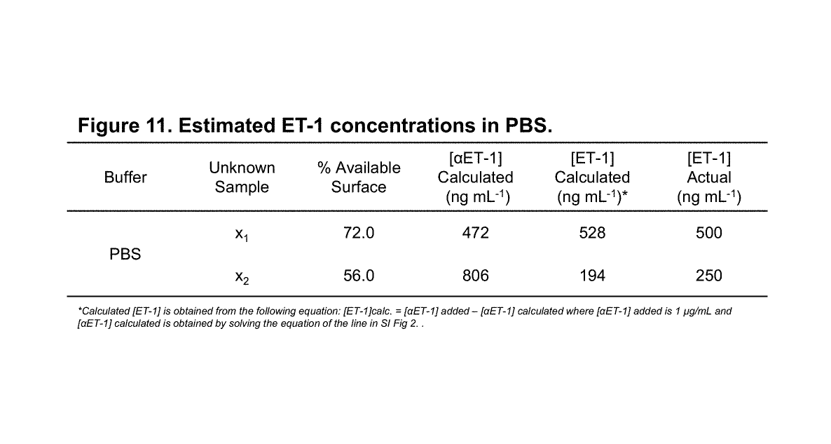

### DETAILED DESCRIPTION

In the following description, numerous specific details are set forth to provide a thorough understanding of the invention. However, it is understood that the invention may be practiced without these specific details.

In an aspect, there is provided a method for the electrochemical quantification of a protein analyte in sample, comprising: providing one or more electrode(s), each comprising at least one peptide attached to its surface, the peptide being the protein or a fragment thereof; contacting the sample and electrode with an antibody in the presence of a redox reporter, wherein the antibody is capable of binding to each of the protein analyte and the peptide on the electrode; measuring an electrochemical signal generated by the redox reporter when a potential is applied; quantifying the protein analyte by comparing the electrochemical signal generated with a control, wherein the electrochemical signal is indirectly proportional to the amount of protein analyte in the sample.

As used herein, “polypeptide” and “protein” are used interchangeably and mean proteins, protein fragments, modified proteins, amino acid sequences and synthetic amino acid sequences, including proteins containing non-naturally occurring amino acids and amino acid analogs, and peptidomimetic structures. The side chains may be in either the (R) or the (S) configuration. In some embodiments, the amino acids are in the (S) or L-configuration. The polypeptide can be glycosylated or not. There are a large number of possible proteinaceous target analytes that may be detected using the present embodiments herein, including proteins containing non-naturally occurring amino acids and amino acid analogs, and peptidomimetic structures.

Exemplary target protein analytes include Endothelin-1 (ET-1), big ET-1, GROα, Vascular cell adhesion protein 1 (VCAM-1), interleukin-1 receptor antagonist (IL-1ra), interleukin 1 beta (IL-1β), IL-6, IL-8, Stem Cell Growth Factor-beta (SCGF-β), Caspase-cleaved cytokeratin 18 fragment (M30), and High mobility group box 1 (HMGB-1). Protein analytes in EVLP test perfusate that are prognostic of transplant outcome are described, for example, in U.S. Patent Publication No. 2015/0377904.

In some embodiments, the target protein analyte is a biomarker whose increased expression in EVLP test perfusate is associated with poor outcome after transplant. In specific embodiments, the target protein analyte is Endothelin-1, a potent vasoconstrictive peptide that plays an important role in lung transplantation. ET-1 expression levels are predictive of transplant outcomes and represent a valuable monitoring tool for surgeons. The methods described herein rapidly measure ET-1 peptide levels in lung perfusate.

### ET-1 (SEQ ID NO. 2)

1 mdyllmifsl lfvacqgape tavlgaelsa vgenggekpt psppwrlrrs krcscsslmd
61 kecvyfchld iiwvntpehv vpyglgsprs kralenllpt katdrenrcq casqkdkkcw
121 nfcgagkelr aedimekdwn nhkkgkdcsk lgkkciyqqlvrgrkirrss eehlrqtrse
181 tmrnsvkssf hdpklkgkps reryvthnra hw

Preferably, the peptide is a fragment of Endothelin-1 (SEQ ID NO. 2), such as the 21 amino acid peptide consisting of the following sequence: CSCSSLMDKE CVYFCHLDIIW (SEQ ID NO. 1). In some embodiments, the electrode is gold and the peptide is bound thereto through the thiol (—SH) moiety of a cysteine residue.

In specific embodiments, the target protein analyte is Growth-Regulated Oncogene-alpha. GROα expression levels are predictive of transplant outcomes and represent a valuable monitoring tool for surgeons. In some embodiments, the peptide is a fragment of GROα, such as the 16 amino acid peptide consisting of the following sequence: CAQTEVIATLKNGRKA (SEQ ID NO: 3).

In specific embodiments, the target protein analyte is Vascular cell adhesion protein 1 (VCAM-1). In some embodiments, the peptide is a fragment of VCAM-1. In some embodiments, the VCAM-1 is modified to add a cysteine residue such that the thiol moiety of the added cysteine residue is bound to the electrode. In some embodiments, the modified VCAM-1 peptide is the 30 amino acid peptide consisting of the following sequence: CVNLIGKNRK EVELIVQEKP FTVEISPGPR (SEQ ID NO: 4).

As used herein, “peptide” is a shorter polypeptide and may refer to peptides less than 1500, 1400, 1300, 1200, 1100, 1000, 900, 800, 700, 600, 500, 400, 300, 200, 150, 100, 90, 80, 70, 60, 50, 40, 30, 20, 10, or 5 amino acids in length, or within ranges bounded by any of the foregoing (i.e. 10-20, 20-30, 10-40 . . . etc.).

The recited antibodies are capable of binding to both the target protein analyte and the peptide bound to the electrode. As a result, the target protein analyte competes with the protein/peptide bound to the electrode for the antibody. Removal of the blocking antibody from the electrode allows the redox reporter to diffuse to the surface of the electrode, resulting in a change in the measured oxidation current.

The terms “antibody” and “immunoglobulin”, as used herein, refer broadly to any immunological binding agent or molecule that comprises a human antigen binding domain, including polyclonal and monoclonal antibodies. Depending on the type of constant domain in the heavy chains, whole antibodies are assigned to one of five major classes: IgA, IgD, IgE, IgG, and IgM. Several of these are further divided into subclasses or isotypes, such as IgG1, IgG2, IgG3, IgG4, and the like. The heavy-chain constant domains that correspond to the different classes of immunoglobulins are termed α, δ, ε, γ and μ, respectively. The subunit structures and three-dimensional configurations of different classes of immunoglobulins are well known.

Generally, where whole antibodies rather than antigen binding regions are used in the invention, IgG and/or IgM are preferred because they are the most common antibodies in the physiological situation and because they are most easily made in a laboratory setting.

The “light chains” of mammalian antibodies are assigned to one of two clearly distinct types: kappa (κ) and lambda (λ), based on the amino acid sequences of their constant domains and some amino acids in the framework regions of their variable domains. There is essentially no preference to the use of κ or λ light chain constant regions in the antibodies of the present invention.

As will be understood by those in the art, the immunological binding reagents encompassed by the term “antibody” extend to all human antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any antibody-like molecule that has an antigen binding region, and this term includes antibody fragments that comprise an antigen binding domain such as Fab′, Fab, F(ab′)₂, single domain antibodies (DABs), T and Abs dimer, Fv, scFv (single chain Fv), dsFv, ds-scFv, Fd, linear antibodies, minibodies, diabodies, bispecific antibody fragments and the like.

The techniques for preparing and using various antibody-based constructs and fragments are well known in the art. Diabodies, in particular, are further described in EP 404, 097 and WO 93/11161.

Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, T and Abs, ds-scFv, dimers, minibodies, diabodies, bispecific antibody fragments and other fragments can also be synthesized by recombinant techniques or can be chemically synthesized. Techniques for producing antibody fragments are well known and described in the art.

The human antibodies or antibody fragments can be produced naturally or can be wholly or partially synthetically produced. Thus the antibody may be from any appropriate source, for example recombinant sources and/or produced in transgenic animals or transgenic plants, or in eggs using the IgY technology. Thus, the antibody molecules can be produced in vitro or in vivo.

Preferably, the human antibody or antibody fragment comprises an antibody light chain variable region (V_{L}) that comprises three complementarity determining regions or domains and an antibody heavy chain variable region (V_{H}) that comprises three complementarity determining regions or domains. Said VL and VH generally form the antigen binding site. The “complementarity determining regions” (CDRs) are the variable loops of β-strands that are responsible for binding to the antigen. Structures of CDRs have been clustered and classified by Chothia et al. (J Mol Biol 273 (4): 927-948) and North et al., (J Mol Biol 406 (2): 228-256). In the framework of the immune network theory, CDRs are also called idiotypes.

As used herein “fragment” relating to a polypeptide or polynucleotide means a polypeptide or polynucleotide consisting of only a part of the intact polypeptide sequence and structure, or the nucleotide sequence and structure, of the reference gene. The polypeptide fragment can include a C-terminal deletion and/or N-terminal deletion of the native polypeptide, or can be derived from an internal portion of the molecule. Similarly, a polynucleotide fragment can include a 3′ and/or a 5′ deletion of the native polynucleotide, or can be derived from an internal portion of the molecule.

Electrodes for the detection systems and methods described herein are any electrically conductive materials with properties allowing linkers on the electrode's surfaces. Electrodes have the capability to transfer electrons to or from a redox reporter and are generally connected to an electronic control and detection device. In general, noble metals, such as, Ag, Au, Ir, Os, Pd, Pt, Rh, Ru and others in their family are suitable materials for electrodes. Noble metals have favorable properties including stability and resistance to oxidation, may be manipulated in various methods such as electrodeposition, and bind to thiols and disulfide containing molecules thereby allowing attachment of said molecules. Other materials can also be used, such as nitrogen-containing conductive compounds (e.g., WN, TiN, TaN) or silicon/silica-based materials, such as silane or siloxane. In certain embodiments, the electrode is gold, palladium or platinum. In other embodiments, the electrode is carbon. In further embodiments, the electrode is indium tin oxide.

In some embodiments, the electrode is a microelectrode. In other embodiments, the microelectrode is a nanostructured microelectrode (“NME”). NMEs are microelectrodes that feature nanostructured surfaces. Surface nanotexturing or nanostructures provide the electrode with an increased surface area, allowing for greater sensitivity, particularly in biosensing applications. Manufacturing of NMEs can be performed via electrodeposition. By varying parameters such as deposition time, deposition potential, supporting electrolyte type and metal ion sources, NMEs of a variety of sizes, morphologies and compositions may be generated. In certain instances, NMEs have a dendritic structure. Complexity of the dendritic structure is achieved by the varying the aforementioned electrodeposition parameters. Exemplary NMEs for use in the systems and methods described herein are described in International Pat. Appl. Ser. No. PCT/CA2009/001212 (published as WO/2010/025547) which is incorporated by reference in its entirety.

Other electrode structures can also be used in the detection systems and methods described herein, including, planar surfaces, wires, tubes, cones and particles. Commercially available macro- and micro-electrodes are also suitable for the embodiments described herein.

Electrodes are sized, for example, from between about 0.0001 to about 5000 microns in length or diameter; between about 0.0001 to about 2000 microns in length or diameter; from between about 0.001 to about 250 microns; from between about 0.01 to about 200 microns; from between about 0.1 to about 100 microns; from between about 1 to about 50 microns; from between about 10 to about 30 microns in length, or below about 10 microns in length or diameter. In certain embodiments, electrodes are sized at about 100 microns, about 30 microns, about 10 microns or about 5 microns in length or diameter. In further embodiments, electrodes are sized at about 8 microns.

In some embodiments, the detection systems and methods described herein, comprise one electrode for detection. In other embodiments, multiple electrodes are used. Use of multiple electrodes can be used in parallel to detect a target analyte via one antibody type attached to each electrode, in some embodiments. Alternatively, in other embodiments, multiple electrodes are used for multiplexing.

In further embodiments, an electrode is located upon a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof.

Substrates can be planar crystalline substrates such as silica based substrates (e.g. glass, quartz, or the like), or crystalline substrates used in, e.g., the semiconductor and microprocessor industries, such as silicon, gallium arsenide, indium doped GaN and the like. Silica aerogels can also be used as substrates, and can be prepared by any known methods. Aerogel substrates may be used as free standing substrates or as a surface coating for another substrate material.

The substrate can take any form and typically is a plate, slide, bead, pellet, disk, particle, microparticle, nanoparticle, strand, precipitate, optionally porous gel, sheets, tube, sphere, container, capillary, pad, slice, film, chip, multiwell plate or dish, optical fiber, etc. The substrate can be any form that is rigid or semi-rigid. The substrate may contain raised or depressed regions on which an assay component is located. The surface of the substrate can be etched using well known techniques to provide for desired surface features, for example trenches, v-grooves, mesa structures, or the like. The substrate can take the form of a photodiode, an optoelectronic sensor such as an optoelectronic semiconductor chip or optoelectronic thin-film semiconductor, or a biochip. The location(s) of electrode(s) on the substrate can be addressable; this can be done in highly dense formats, and the location(s) can be microaddressable or nanoaddressable. In some embodiments, the electrode(s) is on a microfabricated chip.

Surfaces on the substrate can be composed of the same material as the substrate or can be made from a different material, and can be coupled to the substrate by chemical or physical means. Such coupled surfaces may be composed of any of a wide variety of materials, for example, polymers, plastics, resins, polysaccharides, silica or silica-based materials, carbon, metals, inorganic glasses, membranes, or any of the above-listed substrate materials.

The substrate and/or its surface is generally resistant to, or is treated to resist, the conditions to which it is to be exposed in use, and can be optionally treated to remove any resistant material after exposure to such conditions.

Accordingly, in some embodiments, the electrode is a noble metal.

In some embodiments, the electrode is carbon.

In some embodiments, the electrode is indium tin oxide.

In some embodiments, the electrode is gold, palladium or platinum.

In some embodiments, the electrode is a nanostructured microelectrode.

In some embodiments, the electrode is less than about 100 microns, about 5 to about 50 microns, or less than about 10 microns; or about 1.6 mm in diameter.

In some embodiments, the electrode has a surface area of between about 6-9×10⁻⁵ cm² and 2×10⁻² cm².

In some embodiments, the electrode is on a microfabricated chip.

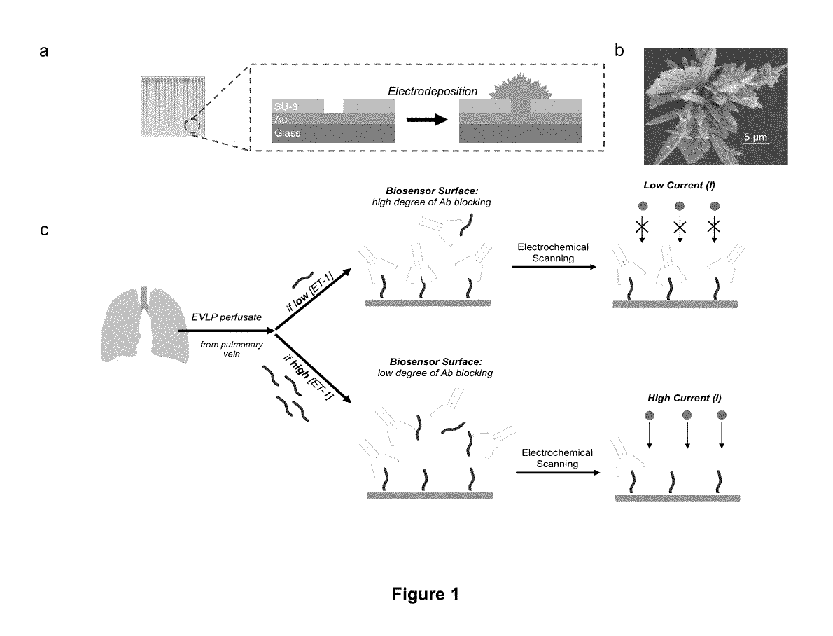

In some embodiments, the microfabricated chip comprises gold, preferably as described in FIG. 1A.

The peptide may be attached in any number of ways to the electrode. The peptide may be attached directly to the electrode. For example, in the case of a gold electrode, the peptide may be attached directly through a cysteine residue on the peptide. Alternatively, the peptide could be attached through a linker or linking chemistry that would be known to a person skilled in the art. Linkers to attach biomolecules to electrodes are described, for example, in U.S. Patent Publication No. 2014/0005068.

Redox reporters suitable for use in the systems and methods described herein are capable of generating an electrical signal (e.g., faradaic current) with the electrode when a potential is applied. Any redox reporter that generates a faradaic current or is capable of interfacial electron transfer with the electrode can be used. Non-limiting redox reporters, include but are not limited to small redox-active groups such as ferricyanide/ferrocyanide, ferrocene and hexachloroiridate(IV)/hexachloroiridate(III). The detection systems utilize redox reporters to generate baseline electrical signals with the electrode.

Samples for the detection systems and methods described herein can be any material suspected of containing an analyte. In some embodiments, the sample can be any source of biological material which comprises proteins that can be obtained from a living organism directly or indirectly, including cells, tissue or fluid, and the deposits left by that organism, including viruses, mycoplasma, and fossils. Typically, the sample is obtained as or dispersed in a predominantly aqueous medium. Nonlimiting examples of the sample include lung perfusate, blood, urine, semen, milk, sputum, mucus, a buccal swab, a vaginal swab, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, samples of in vitro cell culture constituents (including but not limited to conditioned medium resulting from the growth of cells in cell culture medium, putatively virally infected cells, recombinant cells, and cell components), and a recombinant library comprising proteins, peptides, and the like.

The sample can be a positive control sample which is known to contain a target analyte. A negative control sample can also be used which, although not expected to contain the analyte, is suspected of containing it (via contamination of one or more of the reagents) or another component capable of producing a false positive, and is tested in order to confirm the lack of contamination by the target analyte of the reagents used in a given assay, as well as to determine whether a given set of assay conditions produces false positives (a positive signal even in the absence of target analyte in the sample).

The sample can be diluted, dissolved, suspended, extracted or otherwise treated to solubilize and/or purify any target analyte present or to render it accessible to reagents which are used in an amplification scheme or to detection reagents. Where the sample contains cells, the cells can be lysed or permeabilized to release the polynucleotides within the cells. One step permeabilization buffers can be used to lyse cells which allow further steps to be performed directly after lysis, for example a polymerase chain reaction.

As used herein, the term “control” refers to a specific value or dataset that can be used to minimize the effects of variables other than the single independent variable. The control is intended to increase the reliability of the results, often through a comparison between control measurements and the other measurements. A control also includes the use of calibrating samples. For example, in some embodiments, there may be provided the use of standard titration curves (and the like) of known concentrations of protein analyte and protein antibody to quantitate the amount of protein analyte in the sample.

In some embodiments, the electrochemical signal is directly inversely proportional to the amount of antibody bound to the peptide.

In some embodiments, the sample is mixed with the antibody prior to the sample-antibody mixture, in conjunction with the redox reporter, is incubated with the electrode. In other embodiments, the sample is contacted with the antibody prior to the sample-antibody mixture being incubated with the electrode, and the redox reporter is subsequently added.

In some embodiments, the control comprises a standard titration curve of known concentrations of protein analyte and protein antibody. Various parameters may be varied to ensure

In some embodiments, the conductive surface area of the one or more electrode(s) are increased or decreased so as to ensure the electrochemical signal generated by the redox reporter falls within a dynamic range of the standard titration curve.

In some embodiments, the concentration of the antibody is increased or decreased so as to ensure the electrochemical signal generated by the redox reporter falls within a dynamic range of the standard titration curve.

In some embodiments, the ratio of peptide to electrode is increased or decreased so as to ensure the electrochemical signal generated by the redox reporter falls within a dynamic range of the standard titration curve.

In some embodiments, one or more of the conductive surface area of the electrode, the antibody concentration, and the peptide to conductive surface area of the electrode ratio is selected to allow for a measurable change in the electrochemical signal between the sample and a control sample containing no protein analyte.

In some embodiments, the protein analyte is a biomarker for a disease, disorder or condition.

In an aspect, there is provided a kit for the electrochemical detection of a protein analyte in sample, the kit comprising: an electrode comprising a peptide attached to its surface, the peptide being the protein or a fragment thereof; an antibody capable of binding to the protein analyte and the peptide on the electrode; a redox reporter; and instructions for use.

In some embodiments, the kit comprises a plurality of different sized substrates. Preferably, each of the different sized substrates contains a progression of electrodes having different sizes or surface areas. Further preferably, each of the different sized substrates contains a progression of electrodes having different sizes or surface areas in two dimensions, and a progression of different ratios of peptide to electrode concentration across one of said dimensions.

In some embodiments, the kit comprises the antibody in a plurality of concentrations.

In some embodiments, the instructions correspond to the methods described herein.

The advantages of the present invention are further illustrated by the following examples. The examples and their particular details set forth herein are presented for illustration only and should not be construed as a limitation on the claims of the present invention.

### EXAMPLES

### Example 1

Methods and Materials

Microchip Fabrication

Microchips were fabricated in-house at the Toronto Nanofabrication Centre (University of Toronto, Toronto, ON) using precoated (5 nm chromium, 50 nm gold, and AZ1600 (positive photoresist)) glass substrates purchased from Telic Company. Standard contact lithography was used to pattern the sensing electrodes and followed by Au and Cr wet etching steps and removal of the positive photoresist etchant mask. SU-8 2002 (negative photoresist) (Microchem Corp.) was then spin-cast (4000 rpm, 40 s) and patterned using contact lithography to create the 5-500 μm circular sensing apertures. Microchips were diced in-house using a standard glasscutter and washed with acetone (Caledon Labs), isopropyl alcohol (Caledon Labs), and then O₂ plasma etched using (Samco RIE System (Samco)).

Biosensor Electroplating

Gold electrodes (planar or three-dimensional) were electrodeposited at room temperature using a Bioanalytical Systems (BASi) epsilon potentiostat with a three-electrode system featuring a Ag/AgCl reference electrode (BASi) and a platinum wire auxiliary electrode. Gold apertures on the glass microchips served as the working electrode and the biosensors were deposited using 0-50 mM HAuCl₄ (Sigma-Aldrich) using D.C. potential amperometry at 0 mV for 0-30 seconds.

Biosensor Functionalization

Synthetic ET-1 peptides were placed on freshly prepared gold electrodes (1-20 μL probe solution volume) in a humidity chamber and the deposition was allowed to occur overnight at room temperature. Electrodes were thoroughly washed with dH₂O then backfilled with 1 mM MCH (Sigma-Aldrich) for at least 2 hours. Electrodes were thoroughly washed before proceeding to hybridization experiments.

Sensor Blocking

The electrode blocking protocol was as follows: experiments were carried out using 20 μL of 0 to 1 μg mL⁻¹ ET-lantibody (ab48251Abcam) in PBS (Invitrogen) or STEEN Solution™ (XVIVO Perfusion) for 30-60 minutes at room temperature. Following hybridization, electrodes were washed thoroughly and prepared for electrochemical measurements.

Electrochemical Measurements

All electrochemical measurements were performed on a Bioanalytical Systems (BASi) epsilon potentiostat with a three-electrode system featuring a Ag/AgCl reference electrode (BASi), a platinum wire auxiliary electrode, and the biosensing electrode serving as the working electrode. Electrodes were incubated in 2.5 mM [Fe(CN)₆]³⁻ and 2.5 mM [Fe(CN)₆]⁴⁻ (Sigma-Aldrich) for 30 seconds then scanned using differential pulse voltammetry from 0 mV to 400 mV.

Enzyme-Linked Immunosorbent Assay (ELISA)

High-binding, 96-well Costar® plates (Corning Life Sciences) were coated with 10 μg mL⁻¹ of synthetic ET-1 peptide in PBS, overnight, at 4° C. Plates were thoroughly washed then blocked with a solution of 1% (w/v) of BSA (Sigma-Aldrich) in PBS for 60 minutes at room temperature and shaken at 500 rpm. For the ET-1 assay, a preincubation of various concentrations of synthetic ET-1 peptide and 1 μg mL⁻¹ of ET-1 antibody was carried out in a separate reaction tube for 45 minutes prior to being added to the ELISA plate for 45 minutes at room temperature and 500 rpm. Plates were subsequently washed and incubated with streptavidin-HRP (Cell Signaling) for 30 minutes at room temperature and 500 rpm. Following washing, 3,3′5,5′-tetramethybenzidine (TMB) (Cell Signaling) was added to each well for 5-15 minutes and protected from light. To stop the reaction, an equal volume of 1.0 N H₂SO₄ was added to each well and the absorbance at 450 nm was read using a (Spectramax M2 (Molecular Devices)).

ET-1 Analysis Assay (EAA)

Varying concentrations of synthetic ET-1 peptide or perfusate samples collected from a donor lung undergoing EVLP were combined in a separate reaction tube with up to 1 μg mL⁻¹ of ET-1 antibody for 45 minutes prior to being added to the biosensing microchip at room temperature. ET-1 concentrations were calculated by extrapolating x-values (anti-ET-1 antibody concentrations) from experimentally derived y-values (% available surface) based on the equation of the line from a standard curve of anti-ET-1 concentration dilutions. The calculated anti-ET-1 concentration was then subtracted from the actual, added, anti-ET-1 concentration to derive the endogenous ET-1 peptide concentration bound to anti-ET-1 antibodies in solution.

Statistical Analysis

Statistical calculations and analysis were carried out using Prism 6 (GraphPad) and SPSS (IBM) software. For all statistical calculations, a P-value of less than 0.05 was considered statistically significant.

Results and Discussion

Therefore, this work sets out to develop a novel sensing approach for small peptides, such as ET-1. The assay is based on a competitive ELISA-like approach, but incorporates an electrochemical detection method that is sensitive, automatable, and has rapid readout properties.

By exploiting the amino acid sequence of the endothelin-1 peptide, an electrochemical assay was developed to monitor the presence of endogenous ET-1 using an approach similar to that of a competitive ELISA (FIG. 1). An electrochemical approach using glass microchips (FIG. 1a) with highly structured gold microelectrodes (FIG. 1b) provided rapid sample-to-answer times (approximately 30 minutes)¹²⁻¹⁴. As a twenty-one amino acid peptide with an N-terminal cysteine residue, ET-1 bears remarkable similarities (length and N-terminal —SH) to probes used in previous nucleic acid sensing strategies^{13, 15, 16}. Gold microelectrodes were functionalized with a synthetic ET-1 self-assembled monolayer (SAM) (FIG. 1c). By introducing an ET-1 specific antibody, ET-1 antibodies bound to the electrode surface were measured using an ferrocyanide/ferricyanide electrochemical reporter assay¹⁷. In brief, a ferrocyanide molecule is sufficiently small to rapidly diffuse to the surface of an modified electrode; however, the presence of a large, blocking protein (ex. ET-1 antibody) can impede the diffusion of ferrocyanide to the electrode surface (FIG. 1c). As a result, the respective oxidation current of the redox reporter is diminished, as shown by signal attenuation during a differential pulse voltammetry (DPV) electrochemical scan.

In samples collected during EVLP, the presence of the ET-1 peptide in solution competes with the surface-bound ET-1 for the binding of ET-1 antibodies (FIG. 1c, lower track). A portion of the antibody was bound to solution ET-1 (endogenous levels) and the remaining portion was bound to the synthetic, surface bound ET-1. The ferrocyanide blocking signals changed due to differential ET-1 antibody levels bound to the electrode surface in the presence of endogenous ET-1. We hypothesized that we could extrapolate the endogenous [ET-1] from the reported values of ET-1 antibodies blocking the electrode surface.

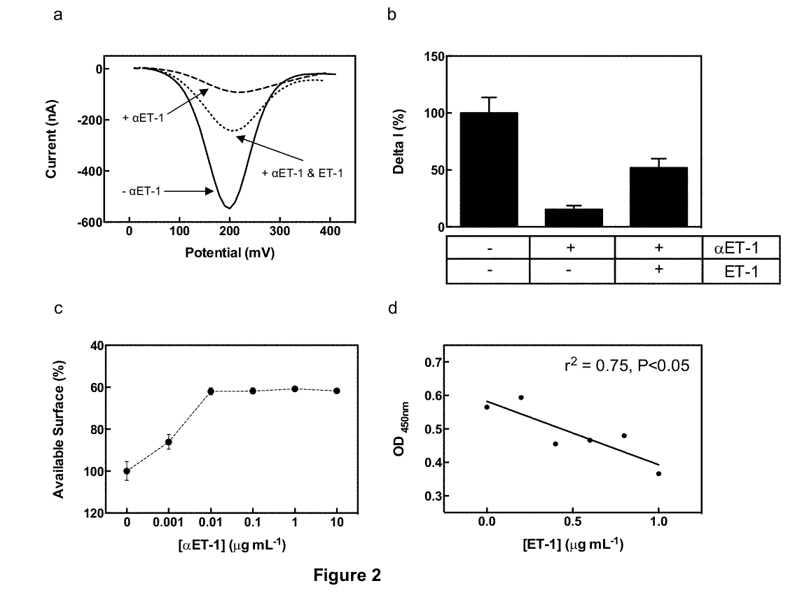

This scheme was validated by comparing the DPV scans of a sensor with no blocking, complete blocking, and partial blocking due to ET-1 peptide in solution (FIG. 2a). A characteristic attenuation of current was observed in the case of complete blocking (FIG. 2a, dashed line) compared to no blocking (FIG. 2a solid line). Where the presence of ET-1 peptide in solution could compete for ET-1 antibodies, only partial blocking was observed (FIG. 2a, dotted line). To quantify the results in FIG. 2a, the relative % blocking of an electrode was calculated to be approximately 85% blocking with no ET-1 peptide in solution compared to 48% blocking with ET-1 in solution (FIG. 2b). The amount of ET-1 antibody detected on the surface of an electrode was titrated (FIG. 2c). A titration profile for the signal attenuation as a function of ET-1 antibody concentration was conducted and dynamic range between 0 and 10 ng mL⁻¹ of antibody (FIG. 2c) was achieved. Above 10 ng mL⁻¹, there was saturation of these sensors. In subsequent proof-of-concept work, sensors that had a higher surface density of synthetic ET-1 peptide (up to 10 μg mL⁻¹) were employed to limit the saturation of the sensors thus improving the dynamic range. To further validate this approach, a competitive ET-1 ELISA was performed in parallel to the electrochemical test (FIG. 2d). As expected, a decrease in the OD_{450 nm} readings were observed as a result of increasing ET-1 peptide concentration (FIG. 2d). The ELISA data matched the electrochemical observations, thus validating the approach; however, the time of the electrochemical assay (1.25 hours) was significantly shorter than that of the ET-1 ELISA assay (4 hours).

In order to expand the dynamic range of this approach, the size of each sensor was varied from small to large by changing the time that the sensors were electrodeposited (30 to 120 seconds) (SI FIG. 1). The degree to which a sensor was blocked (% available surface) was a product of the electrode size. The largest sensors (120 seconds) exhibited limited antibody concentration dependence followed by the medium (60 seconds) and small (30 seconds) sized sensors (SI FIG. 1). In the case of the smallest sensors (30 seconds), an LOD in the range of 10-100 pg mL⁻¹ of ET-1 antibody (SI FIG. 1) was observed. Thus, the ability of the ET-1 assay to be fine-tuned to specific ET-1 peptide levels by altering the size of the biosensing electrodes was confirmed.

A proof-of-concept study of the ET-1 analysis assay (EAA) was performed to validate its accuracy. A standard titration of ET-1 antibody concentrations (SI FIG. 2, closed circles) was run alongside two samples containing spiked concentrations of ET-1 peptide. From the ET-1 antibody titration, linear regression was used to derive an equation representing the antibody concentration as a function of surface blocking (equation shown in upper left-hand corner of SI FIG. 2). From this equation, a theoretical antibody concentration was calculated from the observed surface blocking effects of the unknown samples (x₁ and x₂) (dashed line represents the extrapolation curves). By calculating the difference between the concentrations of the known loaded antibody (1 pg mL⁻¹) to the experimentally observed antibody concentration, the solution-based concentration of ET-1 in the samples were estimated. Using the EAA approach, an average error of 14% was observed (SI Table 1)). Having met the technical requirements of a specific and rapid peptide detection assay, the ET-1 assay was biologically validated in lung perfusate media.

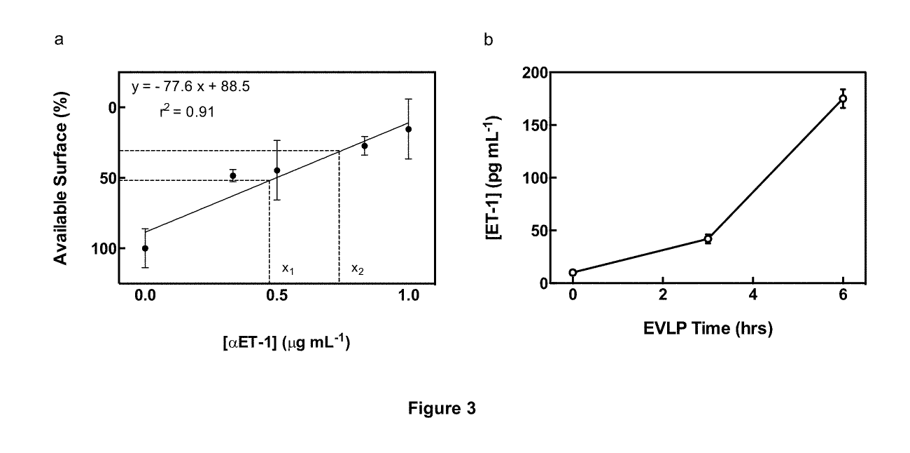

EVLP perfusate solution, STEEN solution, is an acellular matrix that represents a simplified medium for rapid and sensitive biological analysis. During EVLP, diagnostic biomarkers such ET-1 are present and can accumulate in STEEN solution^{2, 18}. Using the same EAA approach as ET-1 detection in PBS, spiked ET-1 levels (500 and 250 ng mL⁻¹) in STEEN solution (FIG. 3a) were tested. ET-1 levels of 526 and 264 ng mL⁻¹ respectively were observed, resulting in an average error of 5.4% in the spiked STEEN assay for the experimentally derived ET-1 concentrations compared to the actual ET-1 concentrations in solution (Table 1). Thus, ET-1 peptide levels in perfusate samples could be extrapolated with a high degree of accuracy. To validate this approach in the transplant setting, perfusate samples collected from a donor lung on EVLP at 3 and 6 hours were tested. Using the EAA, ET-1 levels could be monitored over time in lung perfusate during the course of EVLP (FIG. 3b). The integrity of the assay was upheld as the complexity of the sampling matrix was increased from PBS to STEEN. This was anticipated as the development of a robust SAM on an electrode provided excellent specificity. Taken together, the EAA met the specificity and rapid turnaround parameters required by LTx surgeons to monitor ET-1 peptide levels in lung transplantation-specific media.

The sensing platform may be capable of detecting very short peptide sequences using a competitive electrochemical assay. This indirect approach serves as a strong foundation for determining endogenous ET-1 concentrations in lung perfusate and may be of benefit to transplant teams for the prediction of patient outcomes. Future work will explore efforts that further improve the speed and subsequent timing of the EAA in order to facilitate its clinical implementation. In addition, studies that are focused on determining and quantifying of an absolute cutoff for the ET-1 levels associated with lung and patient outcomes will be investigated. By monitoring ET-1 levels during the transplant process, a new level of biomarker-based patient survival prediction is now possible and this information may be used to guide transplant teams towards targeted therapeutic strategies that, together, may improve the quality of life for the transplant patient.

### Example 2

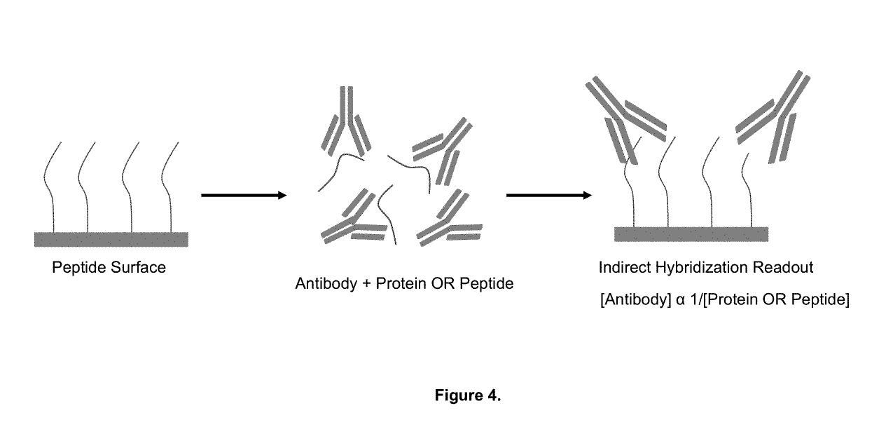

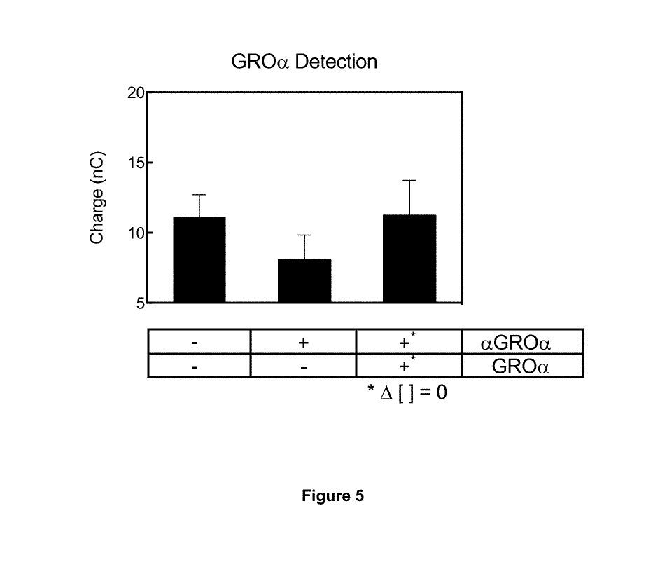

The system and methods of Example 1 were adapted for other analytes. GROα was used as a model analyte (FIG. 4). The GROα peptide having an amino sequence set forth in SEQ ID NO: 3, which includes a thiol residue, was used to functionalize prepared gold electrodes. The GROα antibody (ab86436; Abcam) was used for sensor blocking. Synthetic samples comprising GROα protein (ab92856, Abcam) were prepared to validate the scheme. A detection profile similar to that of ET-1 (FIG. 5) was developed.

### Example 3

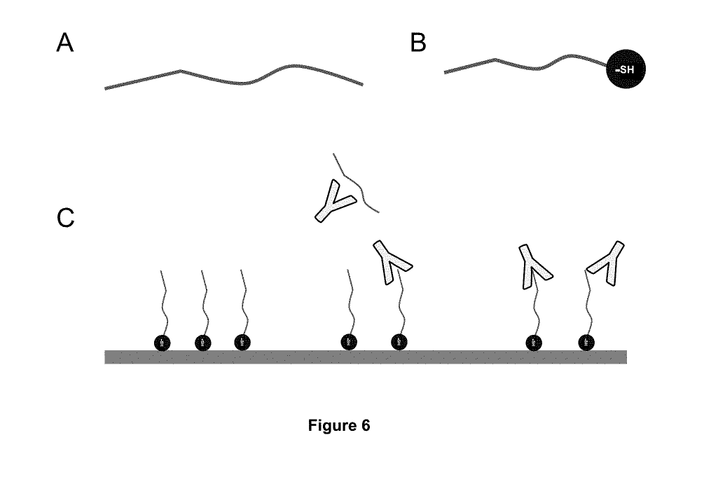

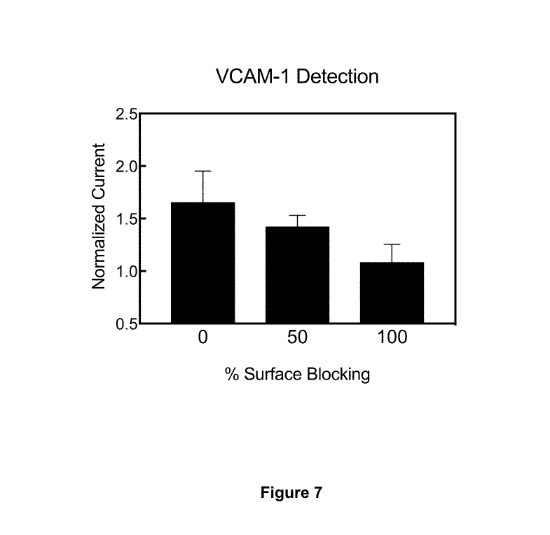

Since not all antigenic peptides of full-length proteins contain a thiol residue, peptides may be modified to allow the utilization of any protein-antibody pair. As a model system, a VCAM-1 peptide-antibody pair was used. The VCAM-1 antigenic peptide (FIG. 6A) was modified to add a thiol moiety (FIG. 6B). The modified VCAM-1 peptide has the amino acid sequence set forth in SEQ ID NO: 4. The VCAM-1 antibody (ab123801; Abcam) was used for sensor blocking. A similar detection scheme to both ET-1 and GROα (FIG. 6C) was employed. Synthetic samples comprising the VCAM-1 peptide were prepared to validate the scheme. The modified peptide allowed for surface modification of the electrode and retained the ability for antibody recognition. Similar trends in VCAM-1 detection in solution as with GROα and ET-1 (FIG. 7) were observed.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. M. Cypel, J. C. Yeung, M. Liu, M. Anraku, F. Chen, W. Karolak, M. Sato, J. Laratta, S. Azad, M. Madonik, C. W. Chow, C. Chaparro, M. Hutcheon, L. G. Singer, A. S. Slutsky, K. Yasufuku, M. de Perrot, A. F. Pierre, T. K. Waddell and S. Keshavjee, N. Engl. J. Med., 2011, 364, 1431-1440.
2. T. N. Machuca, M. Cypel, Y. Zhao, H. Grasemann, F. Tavasoli, J. C. Yeung, R. Bonato, M. Chen, R. Zamel, Y. M. Chun, Z. Guan, M. de Perrot, T. K. Waddell, M. Liu and S. Keshavjee, J. Heart Lung Transplant., 2015, DOI: 10.1016/j.healun.2015.01.003.
3. A. H. Chester and M. H. Yacoub, Glob. Cardiol. Sci. Pract., 2014, 2014, 62-78.
4. M. Uguccioni, L. Pulsatelli, B. Grigolo, A. Facchini, L. Fasano, C. Cinti, M. Fabbri, G. Gasbarrini and R. Meliconi, J. Clin. Pathol., 1995, 48, 330-334.
5. H. Matsumoto, N. Suzuki, H. Onda and M. Fujino, Biochem. Biophys. Res. Commun., 1989, 164, 74-80.
6. M. E. Ivey, N. Osman and P. J. Little, Atherosclerosis, 2008, 199, 237-247.
7. S. E. Verleden, E. Vandermeulen, D. Ruttens, R. Vos, A. Vaneylen, L. J. Dupont, D. E. Van Raemdonck, B. M. Vanaudenaerde and G. M. Verleden, Semin. Respir. Crit. Care Med., 2013, 34, 352-360.
8. D. O. Taylor, L. B. Edwards, M. M. Boucek, E. P. Trulock, P. Aurora, J. Christie, F. Dobbels, A. O. Rahmel, B. M. Keck and M. I. Hertz, J. Heart Lung Transplant., 2007, 26, 769-781.
9. M. Salama, P. Jaksch, O. Andrukhova, S. Taghavi, W. Klepetko and S. Aharinejad, J. Thorac. Cardiovasc. Surg., 2010, 140, 1422-1427.
10. M. Salama, O. Andrukhova, P. Jaksch, S. Taghavi, W. Kelpetko, G. Dekan and S. Aharinejad, Transplantation, 2011, 92, 155-162.
11. G. I. Snell, M. Rabinov, A. Griffiths, T. Williams, A. Ugoni, R. Salamonsson and D. Esmore, J. Heart Lung Transplant., 1996, 15, 160-168.
12. B. Lam, J. Das, R. D. Holmes, L. Live, A. Sage, E. H. Sargent and S. O. Kelley, Nat. Commun., 2013, 4, 2001.
13. B. Lam, Z. Fang, E. H. Sargent and S. O. Kelley, Anal. Chem., 2012, 84, 21-25.
14. L. Soleymani, Z. Fang, B. Lam, X. Bin, E. Vasilyeva, A. J. Ross, E. H. Sargent and S. O. Kelley, ACS Nano, 2011, 5, 3360-3366.
15. Z. Fang, L. Soleymani, G. Pampalakis, M. Yoshimoto, J. A. Squire, E. H. Sargent and S. O. Kelley, ACS Nano, 2009, 3, 3207-3213.
16. E. Vasilyeva, B. Lam, Z. Fang, M. D. Minden, E. H. Sargent and S. O. Kelley, Angew. Chem. Int. Ed. Engl., 2011, 50, 4137-4141.
17. J. Das and S. O. Kelley, Anal Chem, 2011, 83, 1167-1172.
18. T. N. Machuca, M. Cypel, J. C. Yeung, R. Bonato, R. Zamel, M. Chen, S. Azad, M. K. Hsin, T. Saito, Z. Guan, T. K. Waddell, M. Liu and S. Keshavjee, Ann. Surg., 2015, 261, 591-597.

1. A method for the electrochemical quantification of a protein analyte in sample, comprising:
a. providing one or more electrode(s), each comprising at least one peptide attached to its surface, the peptide comprising the protein or a fragment thereof;
b. contacting the sample and electrode with an antibody in the presence of a redox reporter, wherein the antibody is capable of binding to each of the protein analyte and the peptide on the electrode;
c. measuring an electrochemical signal generated by the redox reporter when a potential is applied;
d. quantifying the protein analyte by comparing the electrochemical signal generated with a control, wherein the electrochemical signal is indirectly proportional to the amount of protein analyte in the sample.

2. (canceled)

3. The method of claim 1, wherein the sample is mixed with the antibody prior to the sample-antibody mixture, in conjunction with the redox reporter, being incubated with the electrode.

4. The method of claim 1, wherein the sample is contacted with the antibody prior to the sample-antibody mixture being incubated with the electrode, and the redox reporter is subsequently added.

5. The method of claim 1, wherein the control comprises a standard titration curve of known concentrations of protein analyte and protein antibody.

6. The method of claim 5, wherein at least one of the conductive surface area of the one or more electrode(s), the concentration of the antibody, and the ratio of peptide to electrode surface area is increased or decreased so as to ensure the electrochemical signal generated by the redox reporter falls within a dynamic range of the standard titration curve.

7. (canceled)

8. (canceled)

9. The method of claim 1, wherein one or more of the conductive surface area of the electrode, the antibody concentration, and the peptide to conductive surface area of the electrode ratio is selected to allow for a measurable change in the electrochemical signal between the sample and a control sample containing no protein analyte.

10. The method of claim 1, wherein the peptide is between 5 and 1500 amino acids in length.

11. The method of claim 1, wherein the redox reporter is ferricyanide/ferrocyanide ferrocene, or hexachloroiridate(IV)/hexachloroiridate(III).

12. (canceled)

13. (canceled)

14. (canceled)

15. The method of claim 1, wherein the electrode is carbon indium tin oxide, gold, palladium or platinum.

16. (canceled)

17. (canceled)

18. (canceled)

19. (canceled)

20. (canceled)

21. (canceled)

22. (canceled)

23. (canceled)

24. (canceled)

25. The method of claim 1, wherein the protein analyte is IL-1ra, SCGF-b, IL-8, big ET-1, IL-6, IL-1B, M30, HMGB-1, ET-1, VCAM-1, GROα, or a combination thereof.

26. The method of claim 25, wherein the protein analyte is Endothelin-1.

27. The method of claim 26, wherein the peptide is a 21 amino acid peptide consisting of the following sequence: CSCSSLMDKECVYFCHLDIIW (SEQ ID NO. 1).

28. The method of claim 26, wherein the peptide is a fragment of Endothelin-1 (SEQ ID NO. 2).

29. The method of claim 25, wherein the protein analyte is GROα.

30. The method of claim 29, wherein the peptide is a 16 amino acid peptide consisting of the following sequence: CAQTEVIATLKNGRKA (SEQ ID NO: 3).

31. The method of claim 25, wherein the protein analyte is VCAM-1.

32. The method of claim 31, wherein the peptide is a 30 amino acid peptide consisting of the following sequence: CVNLIGKNRKEVELIVQEKPFTVEISPGPR (SEQ ID NO:4).

33. (canceled)

34. (canceled)

35. The method of claim 1, wherein the sample comprises lung perfusate.

36. The method of claim 1, wherein the electrode is gold and the peptide is bound thereto through a thiol moiety of the peptide.

37. (canceled)

38. (canceled)

39. (canceled)

40. (canceled)

41. (canceled)

42. (canceled)

43. (canceled)

44. (canceled)
